(19) United States
(12) Patent Application Publication
(10) Pub. No.: US 2004/0250845 A1
(43) Pub. Date: Dec. 16, 2004

(54) Walking stick with flexure mechanism to store and release energy

(76) Inventors: Neal H. Rudin, Rochester, NY (US); Joseph D. LaRussa, Rochester, NY (US)

Correspondence Address: Garold F. Fritz, 4325 Lake Road, Williamson, NY, 14589-9603, US

(21) Appl. No.: 10/865,052

(22) Filed: Jun. 10, 2004

Related U.S. Application Data
(60) Provisional application No. 60/477,913, filed on Jun. 13, 2003.

(51) Int. Cl. A61F 2/66; A61H 3/02; A45B 9/04

(52) U.S. Cl. 135/77

(57) ABSTRACT

A walking stick with a flexure spring to store energy from compression during the user's step, and release of the energy to aid in propelling the user forward, thereby reducing fatigue and enabling longer and faster walks.

### CROSS-REFERENCE TO RELATED APPLICATIONS

This application, excluding claims 10 and 20, claims the benefit of U.S. Provisional Application No. 60/477,913 filed Jun. 13, 2003.

### FIELD OF THE INVENTION

The invention relates in general to walking aid sticks and, more specifically, to a walking stick with a built in flexure spring to return energy into each step to help propel the user forward.

### BACKGROUND OF THE INVENTION

Walkers and climbers can benefit from walking sticks that have the feature of returning kinetic energy that is acquired as the walking stick compresses in contact with the ground. This retained energy has the benefit that the users have to expend less of their own energy moving their legs forward, thus allowing them to walk or climb further and faster more comfortably and with less fatigue. Experiencing this advantage, the user will increase their interest in the activity and become a more active person.

The use of a walking stick with an energy storage spring for returning energy to the walker is well known to those familiar with the art. An example is the use of energy storage springs in foot prosthesis; particularly those used for athletic purposes. Designs include that of U.S. Pat. No. 6,007,582 or that in use in the Flex-foot®, manufactured by Ossur hf of Reykjavik, Iceland. Examples where a spring mechanism stores and then sequentially dissipates energy for the sole purpose of cushioning a walking stick are described in U.S. Pat. No. 6,131,592, U.S. Pat. No. 5,720,474, and FR2617023. These walking sticks utilize a mechanical device such as a coil spring or cylinder for a spring and do not make use of the compression of a flexure to return the energy from the material compressing as a step is taken.

A published paper entitled “The design of a compliant composite crutch” by D. Shortell et al. discloses two designs of crutches using composite materials. The first design utilizes a metal coil spring embedded in a single unit composite material crutch. The coil spring compresses under the weight of the user with a spring force in the range of 90 to 170 lbs. The spring force, which acts in the vertical direction, is for shock absorption, not as a forward propelling aid. A second crutch design utilizes the flexure of the S curve in the shaft of the crutch in place of the coil spring. The effective springs that are designed using the composite material in place of the coil spring also operate in the vertical direction for shock absorption, not as a forward propelling aid. Another feature of the crutches that are the subject of the Shortell et al. publication is a rigid armrest with a grip. These armrests are oriented in the vertical direction for the purpose of providing the user with more support.

### SUMMARY OF THE INVENTION

In light of the above, the object of the present invention is to provide a walking stick that absorbs energy in the downward first motion of a walking stride and then returns the stored energy to aid in propelling the walker forward in the final forward motion of the walking stride.

The walking stick that is the subject of this patent application utilizes a flexure spring as an extension of the straight shaft of the stick. The shape and location of the flexure spring are such that the spring force helps propel the walker forward. The spring constant of the flexure spring is in the range of 5 to 50 lbs. per inch of deflection. In the preferred embodiment the flexure spring is fabricated using composite materials.

The walking stick of the present invention may be used in a wide number of applications. Examples include a walking and climbing stick for hikers, an ambulatory aid for a person recovering from surgery or otherwise limited in ability to walk, a substitute for a ski pole for cross country skiing, a pole for use in roller blading, a hiking stick that will also function as a canoe paddle, or a walking stick for snow shoeing. It may be a molded single unit or assembled out of multiple components. As a molded single unit the flexure spring is integral to the molded stick. As a stick built of multiple components, the flexure spring is attached to the straight shaft of the stick and may be interchangable depending upon the size and weight of the user, or depending on one of the specific uses listed above.

The invention, and its objects and advantages, will become more apparent in the detailed description of the preferred embodiment presented below.

### BRIEF DESCRIPTION OF THE DRAWINGS

In the detailed description of the preferred embodiments of the invention presented below, reference is made to the accompanying drawings, in some of which the relative relationships of the various components are illustrated, it being understood that orientation of the apparatus may be modified. For clarity of understanding of the drawings, relative proportions depicted or indicated of the various elements of which disclosed members are comprised may not be representative of the actual proportions, and some of the dimensions may be selectively exaggerated.

FIG. 1 illustrates a single-piece walking stick.

FIG. 2 illustrates a multi-piece walking stick.

FIG. 3 illustrates a multi-piece walking stick with adjustable length.

FIG. 4 illustrates a walking stick with a horizontal arm support.

FIG. 5 is plot of force versus deflection for two embodiments of walking sticks of the present invention.

### DETAILED DESCRIPTION OF THE INVENTION

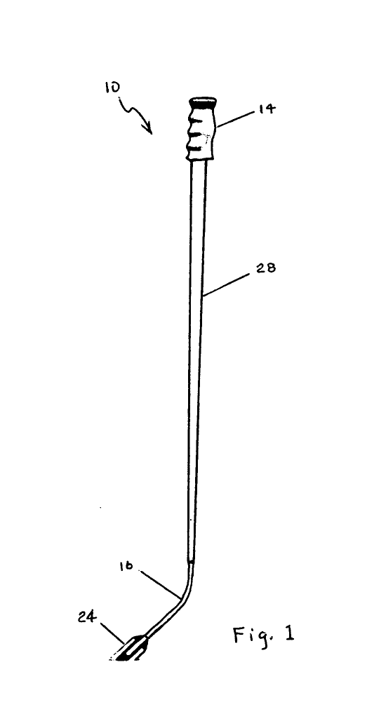

Referring to FIG. 1, therein is shown a preferred embodiment of the invention. This walking stick, generally designated by numeral 10, is molded as a single unit of fiberglass, carbon, or other composite material. Walking stick 10 includes a straight shaft 28 terminated at the upper end with a handgrip 14 and at the lower end with a flexure spring 16. Walking stick 10 may be constructed of laminated linear fiberglass or other composite material. In a preferred embodiment walking stick 10 is constructed of a fiberglass tape such as that manufactured by the Fibreglast Development Corp. of Brookville, Ohio, USA. Attached to flexure spring 16, at the point where flexure spring 16 would contact the walking surface, is a foot 24. The purpose of foot 24 is to prevent flexure spring 16 from sliding on the walking surface. Foot 24 may be made from rubber, such as, for example, a section of rubber tubing, or any other material with a suitable coefficient of friction with the walking surface.

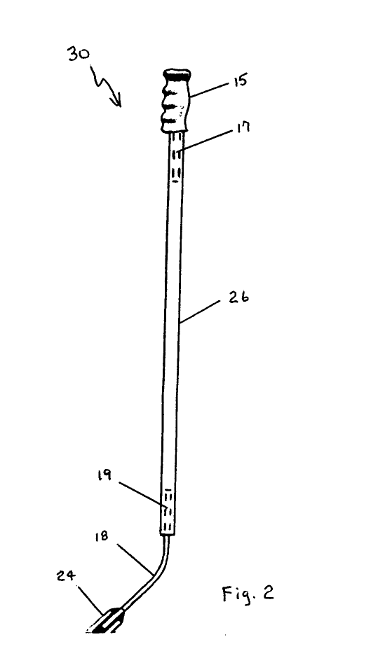

Another embodiment of the walking stick of the present invention is shown in FIG. 2 and designated generally as numeral 30. Walking stick 30 in FIG. 2 includes similar sections as walking stick 10 in FIG. 1, that is, a straight shaft 26, a handgrip 15, and a flexure spring 18, but the sections of walking stick 30 are separate assembled parts instead of molded as a single unit. In the walking stick 30 embodiment flexure spring 18 is constructed of laminated linear fiberglass or other composite material. Straight shaft 26 may also be constructed of laminated linear fiberglass or other composite material or of any other rigid material such as, for example, aluminum, steel, stainless steel, or the like. Handgrip 15 is constructed of hard rubber, wood, or any other similar material. Flexure spring 18 and handgrip 15 are connected to opposite ends of straight shaft 26 by pins 19 and 17 respectively. Flexure spring 18 of the walking stick 30 embodiment also has attached to it a foot 24, the same as in the walking stick 10 embodiment.

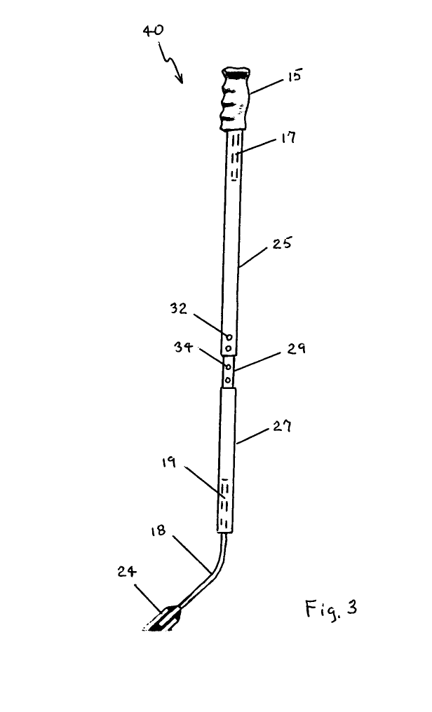

For some sports applications an adjustable length walking stick is preferred. Referring to FIG. 3, therein is shown a design of an adjustable length walking stick 40. The walking stick 40 embodiment has the same flexure spring 18 with pin 19, and handgrip 15 with pin 17 as the walking stick 30 embodiment of FIG. 2. The straight shaft of the walking stick 40 embodiment includes two telescoping sections 25 and 27. Several means for locking the adjusted length of sections 25 and 27 are known in the art, one of which is shown in FIG. 3. Section 25 contains a spring-loaded pin 32 that is pushed into one of a series of holes 34 in section 26 to secure the desired length of walking stick 40. Other adjustable means can be used such as the mechanism cited in U.S. Pat. No. 5,769,104.

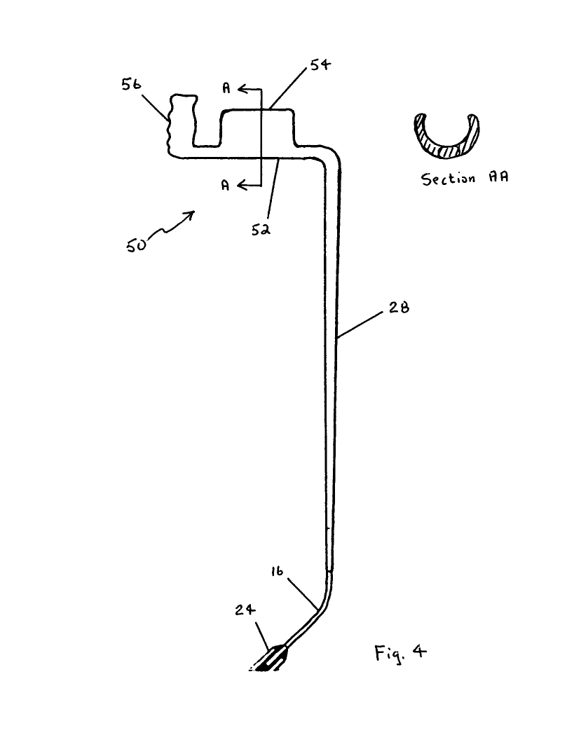

FIG. 4 shows another embodiment of the walking stick of the present invention generally designated by numeral 50. Walking stick 50 embodiment is distinguished by an arm support section 52 at an approximate 90° angle to the vertical straight shaft 28. Arm support section 52 has a semi-cylindrical arm support 54 and a handgrip 56. AA is a cross-section view of semi-cylindrical arm support 54. Straight vertical shaft 28 and flexure spring 16 with foot 24 in the walking stick 50 embodiment are the same is in the walking stick embodiment 10 of FIG. 1. Arm support section 52, with semi-cylindrical arm support 54, of the walking stick 50 embodiment is positioned in the horizontal direction, in line with the natural arm and hand posture used when walking or hiking and helps reduce arm fatigue on long hikes and especially climbing. Arm support section 52 with semi-cylindrical arm support 54 and handgrip 56 may also replace handgrip 15 of the walking stick embodiments of FIGS. 2 and 3, and be molded from plastic. To provide protection for the walker's hands, handgrip 56 of the walking stick 50 embodiment may also be surrounded by a molded hand guard.

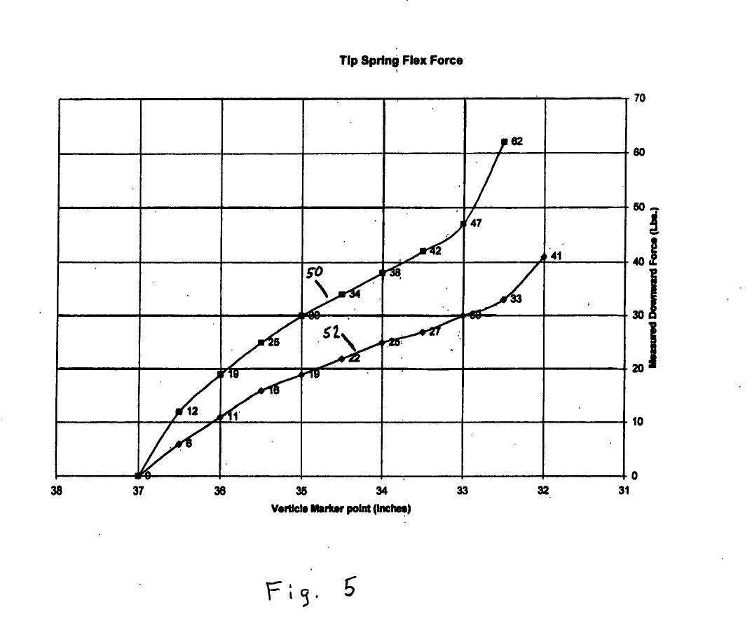

FIG. 5 illustrates, for two preferred embodiments of the walking stick of the present invention, the amount of spring force as a function of the amount of deflection of the flexure spring. Curve 50 represents the result for a preferred embodiment walking stick for a person weighing in the range of 175 to 225 lbs. Curve 52 represents the characteristics of a preferred embodiment hiking stick for a person weighing in the range of approximately 100 lbs. Curve 50 indicates that a deflection of 1 inch produces a spring force of 19 lbs. Similarly curve 52 indicates that a deflection of 1 inch produces a spring force of 10 to 11 lbs. These curves show that with a deflection greater than 4 inches, the relationship between deflection and force increases nonlinearly so that when the walking stick is heavily compressed, large spring forces result. When the transition to a higher spring force is reached, it is a signal to the athlete to push off or, in other words, to transfer his/her weight to the other stick.

The multi-part embodiments of the walking stick of the present invention, as illustrated in FIGS. 2 and 3, enable the use of interchangable flexure springs with different spring force constants for different weight users, or with different shaped feet for use on different surfaces. For example, the foot shown in FIGS. 14 may be optimum for use on hard packed or paved surfaces, whereas a flared, higher surface area foot may be better for use in snow or mud. An extremely flared foot with very high surface area may serve double duty as a walking stick and as a canoe paddle.

The invention has been described in detail with particular reference to certain preferred embodiments thereof, but it will be understood that variations and modifications can be effected within the spirit and scope of the invention.

What is claimed is:

1. A walking aid comprising, a straight shaft terminated with a handgrip at an upper end, and with a curvilinear shaped flexure spring at a lower end, said curvilinear shaped flexure spring having a free end, distal from said straight shaft, so that said free end will contact the surface upon which a person, gripping said handgrip, is standing, said free end having an attached foot providing means for preventing said free end from sliding on the surface upon which said person is standing.

2. The walking aid of claim 1 wherein said curvilinear shaped flexure spring has a spring constant in the range between 5 and 50 pounds/inch.

3. The walking aid of claim 2 wherein said straight shaft, said handgrip, and said curvilinear shaped flexure spring are integrally formed in a single part of a common material.

4. The walking aid of claim 3 wherein said common material comprises a plurality of layers of a composite material.

5. The walking aid of claim 4 wherein said composite material is chosen from the group consisting of fiberglass and carbon fiber.

6. The walking aid of claim 2 wherein said straight shaft, said handgrip, and said curvilinear shaped flexure spring are separate parts with means for connecting together.

7. The walking aid of claim 6 wherein said curvilinear shaped flexure spring comprises a plurality of layers of a composite material.

8. The walking aid of claim 7 wherein said composite material is chosen from the group consisting of fiberglass and carbon fiber.

9. The walking aid of claim 8 wherein said straight shaft further comprises an upper section and a lower section with means for connecting said upper section to said lower section so that the overall length is adjustable.

10. The walking aid of claim 6 wherein said curvilinear shaped flexure spring is made from a material selected from the group consisting of a metal, a wood, a plastic, and a ceramic.

11. A walking aid comprising, an L-shaped shaft with a generally horizontal upper section at approximately 90° to a generally vertical lower section, said upper section having a semi-cylindrical arm support, and terminated with a handgrip, said lower section terminated with a curvilinear shaped flexure spring, said curvilinear shaped flexure spring having a free end, distal from said generally vertical lower section, so that said free end will contact the surface upon which a person, gripping said handgrip, is standing, said free end having an attached foot providing means for preventing said free end from sliding on the surface upon which said person is standing.

12. The walking aid of claim 11 wherein said curvilinear shaped flexure spring has a spring constant in the range between 5 and 50 pounds/inch.

13. The walking aid of claim 12 wherein said L-shaped shaft, said handgrip, and said curvilinear shaped flexure spring are integrally formed in a single part of a common material.

14. The walking aid of claim 13 wherein said common material comprises a plurality of layers of a composite material.

15. The walking aid of claim 14 wherein said composite material is chosen from the group consisting of fiberglass and carbon fiber.

16. The walking aid of claim 12 wherein said L-shaped shaft, said handgrip, and said curvilinear shaped flexure spring are separate parts with means for connecting together.

17. The walking aid of claim 16 wherein said curvilinear shaped flexure spring comprises a plurality of layers of a composite material.

18. The walking aid of claim 17 wherein said composite material is chosen from the group consisting of fiberglass and carbon fiber.

19. The walking aid of claim 18 wherein said L-shaped shaft further comprises an upper section and a lower section with means for connecting said upper section to said lower section so that the overall length is adjustable.

20. The walking aid of claim 16 wherein said curvilinear shaped flexure spring is made from a material selected from the group consisting of a metal, a wood, a plastic, and a ceramic.
